(19) United States
(12) Patent Application Publication
(10) Pub. No.: US 2022/0215967 A1
(43) Pub. Date: Jul. 7, 2022

(54) Quantum Computing System

(71) Applicant: Hong Kong Applied Science And Technology Research Institute Co., Ltd., Shatin (HK)

(72) Inventors: Xiaoyu Zhao, Pak Shek Kok (HK); Zhibin Lei, Pak Shek Kok (HK); Hang Liu, Xi'an city (HK); Yang Cai, Shenzhen City (HK)

(21) Appl. No.: 17/140,253

(22) Filed: Jan. 4, 2021

(51) Int. Cl. G16H 50/80; G06N 10/00; G06F 17/13

(52) U.S. Cl. CPC G16H 50/80; G06F 17/13; G06N 10/00

(57) ABSTRACT

Described is a computing system comprising: a quantum computer comprising a quantum computer controller; and one or more quantum processors; and a classical computer comprising: a memory storing machine-readable instructions; and a processor for executing the machine-readable instructions. When the processor executes the machine-readable instructions, it configures the classical computer to model a physical system; and to implement the steps in said computing system of: transforming a plurality of non-linear equations into differential equations whose non-linear terms are defined by polynomials; encoding the polynomials as probability amplitudes of a quantum state of a quantum system; evolving the quantum system into a new quantum system comprising message qubits and at least one ancilla qubit; and utilizing a quantum iterative optimization algorithm to solve the plurality of differential equations. The at least one ancilla qubit can be measured to determine or calculate a value for one of the dependent variables with respect to the at least one independent variable. At least the utilizing step is performed by the one or more quantum processors.

### FIELD OF THE INVENTION

The invention relates to a quantum computing system and a method implemented by said system to calculate or determine values for dependent variables of a real, modelled or simulated physical or real-world system. The invention is more particularly related to a Susceptible-Infectious-Recovered (SIR) computing system for determining values of variables related to epidemics.

### BACKGROUND OF THE INVENTION

As the size of semiconductors approaches the nanometer (nm) level, quantum effects start to play significant roles in semiconductor devices such as, for example, processors. Quantum processors can perform certain tasks much more efficiently and quickly than classical computers. Classical computers that are used today can only encode information in bits that take the binary values 1 or 0. This considerably restricts their processing capacity.

Quantum processors, on the other hand, use quantum bits or “qubits”. A quantum processor harnesses the unique ability of subatomic particles that allows them to exist in more than one state at the same time, i.e., to exist in superimposed states taking the values 1 and 0 at the same time. In general, the classical computer is good at calculus. On the other hand, the quantum processor is much better at calculations, sorting data, finding prime numbers, simulating molecules, and optimization processes, etc. In other words, quantum processors with a modest number of qubits can perform calculations which would otherwise require a classical supercomputer.

Many physical, real-world systems can be defined, modelled, or simulated on the basis that they can be defined by at least one independent variable, a plurality of dependent variables and a plurality of parameters associated with said plurality of dependent variables, where said plurality of dependent variables is defined by a plurality of non-linear equations with each non-linear equation being based on at least one of the plurality of parameters.

Infectious diseases are a major cause of death worldwide and have in the past killed many more people than in all wars throughout history. Mathematical modelling of infectious diseases was initiated by Bernoulli in 1760. The work of Kermack and McKendrick, published in 1927, had a major influence on the modelling framework. Their Susceptible-Infectious-Recovered (SIR) model is still used to model epidemics of infectious diseases. The SIR model tracks the numbers of susceptible, infected, and recovered individuals in one or more populations during an epidemic with the help of ordinary differential equations.

The SIR model is an example of a model of a physical or real-world system in the context of this invention in that it models the changing values of the dependent variables of the epidemic with respect to the independent variable time for a population. In this instance, the population subjected to the epidemic comprises the physical or real-world system.

Since the calculation of these ordinary differential equations is complex and time consuming when using classical computers, it is desired to provide a quantum computing system embodying, in one embodiment, a Quantum SIR (QSIR) model to reduce the computational complexity and time to results, and which can be used to simulate the real physical system and make determinations, calculations and predictions of future values of one or more of the dependent variables of such system.

### OBJECTS OF THE INVENTION

An object of the invention is to mitigate or obviate to some degree one or more problems associated with known methods of calculating values of dependent variables with respect to one or more independent variables in a physical or real-world system.

The above object is met by the combination of features of the main claims; the sub-claims disclose further advantageous embodiments of the invention.

Another object of the invention is to provide a quantum computing system embodying a quantum model of a physical or real-world system.

Another object of the invention is to provide a quantum computing system embodying a QSIR model for epidemics.

One skilled in the art will derive from the following description other objects of the invention. Therefore, the foregoing statements of object are not exhaustive and serve merely to illustrate some of the many objects of the present invention.

### SUMMARY OF THE INVENTION

Generally, the invention provides a computing system comprising: a quantum computer comprising one or more quantum computer processors or controllers and one or more quantum registers; and a classical computer comprising: a memory storing machine-readable instructions, and a processor for executing the machine-readable instructions. When the processor executes the machine-readable instructions, it configures the classical computer to model a physical system; and to implement steps in said computing system of: transforming a plurality of non-linear equations into differential equations whose non-linear terms are defined by polynomials; encoding the polynomials as probability amplitudes of a quantum state of a quantum system; evolving the quantum system into a new quantum system comprising message qubits and at least one ancilla qubit; and utilizing a quantum iterative optimization algorithm to solve the plurality of differential equations. Then at least one ancilla qubit can be measured to determine or calculate a value for at least one of the dependent variables with respect to at least one independent variable. At least the utilizing step is performed by the one or more quantum processors.

In a first main aspect, the invention provides a method of determining or calculating a value of a dependent variable with respect to a value of an independent variable in a physical system defined or modelled by at least one independent variable, a plurality of dependent variables and a plurality of parameters associated with said plurality of dependent variables, said plurality of dependent variables being defined by a plurality of non-linear equations, each non-linear equation being based on at least one of the plurality of parameters, the method comprising the steps of: transforming the plurality of non-linear equations into differential equations whose non-linear terms are defined by polynomials; encoding the polynomials as probability amplitudes of a quantum state of a quantum system; evolving the quantum system into a new quantum system comprising message qubits and at least one ancilla qubit; utilizing a quantum iterative optimization algorithm to solve the plurality of differential equations; optionally performing a reverse phase estimation operation on the message qubits; and measuring the at least one ancilla qubit to determine or calculate a value for one of the dependent variables with respect to the at least one independent variable.

In a second main aspect, the invention provides a computing system comprising: a quantum computer comprising: one or more quantum processors and one or more quantum registers; a classical computer comprising: a memory storing machine-readable instructions; and a processor for executing the machine-readable instructions such that, when the processor executes the machine-readable instructions, it configures the classical computer to model a physical system and to implement the steps in said computing system of the first main aspect of the invention, wherein the at least the utilizing step is performed by the one or more quantum processors.

In a third main aspect, the invention provides a non-transitory computer-readable medium storing machine-readable instructions, wherein, when the machine-readable instructions are executed by a processor of a computing system according to the second main aspect of the invention, it causes said computing system to implement the steps of the first main aspect of the invention.

The summary of the invention does not necessarily disclose all the features essential for defining the invention; the invention may reside in a sub-combination of the disclosed features.

The forgoing has outlined fairly broadly the features of the present invention in order that the detailed description of the invention which follows may be better understood. Additional features and advantages of the invention will be described hereinafter which form the subject of the claims of the invention. It will be appreciated by those skilled in the art that the conception and specific embodiment disclosed may be readily utilized as a basis for modifying or designing other structures for carrying out the same purposes of the invention.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 is a schematic block diagram of a computing system in accordance with the invention; and

FIG. 2 is a quantum gate diagram illustrating significant steps of the method in accordance with the invention.

### DESCRIPTION OF PREFERRED EMBODIMENTS

The following description is of preferred embodiments by way of example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

It should be understood that the elements shown in the FIGS, may be implemented in various forms of hardware, software or combinations thereof. These elements may be implemented in a combination of hardware and software on one or more appropriately programmed general-purpose devices, which may include a processor, memory, and input/output interfaces.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein represent conceptual views of systems and devices embodying the principles of the invention.

The functions of the various elements shown in the figures may be provided by dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

In the claims hereof, any element expressed as a means for performing a specified function is intended to encompass any way of performing that function including, for example, a) a combination of circuit elements that performs that function or b) software in any form, including, therefore, firmware, microcode, or the like, combined with appropriate circuitry for executing that software to perform the function. The invention as defined by such claims resides in the fact that the functionalities provided by the various recited means are combined and brought together in the manner which the claims call for. It is thus regarded that any means that can provide those functionalities are equivalent to those shown herein.

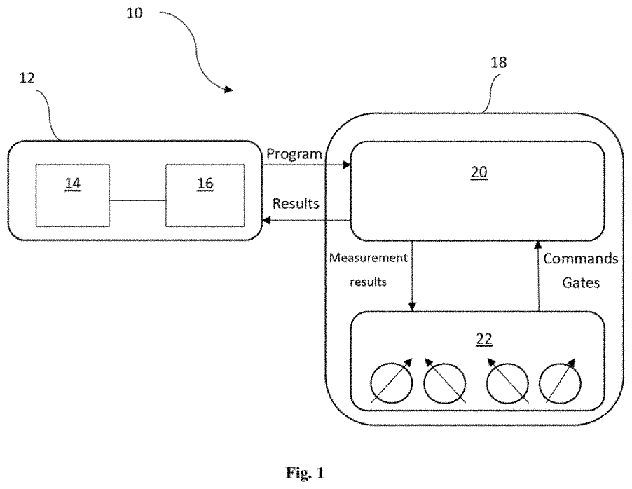

Referring to FIG. 1, provided is a schematic block diagram of a quantum computing system 10 in accordance with the invention. The computing system 10 comprises a classical computer 12 comprising a memory 14 storing machine-readable instructions and a processor 16 for executing the machine-readable instructions. The classical computer 12 could comprise a personal computer or the like. The computing system 10 also comprises a quantum computer 18 comprising one or more quantum computer controllers or processors 20 and one or more quantum registers 22.

The quantum computing system 10 therefore comprises both classical and quantum parts. The machine-readable instructions comprise a classical program stored in the memory 14 of the classical computer 12. A quantum iterative optimization algorithm is provided, as explained more fully below, which uses both classical and quantum data.

The quantum computer 18 can be considered as providing a quantum co-processor to the classical computer 12 where the quantum computer 18 is configured to perform specific tasks in the context of the classical program stored in the memory 14 of the classical computer 12.

The one or more quantum registers 22 hold the quantum data for the quantum iterative optimization algorithm. In one embodiment, the one or more quantum registers 22 sit still, and the quantum operations are executed on the quantum data where they sit. In another embodiment, the quantum computer 18 moves individual atoms around, but not far, so that it can still be considered in terms of a register being in a specific place. An exception to this is quantum computers that use photons for qubits as the photons travel at the speed of light.

The one or more quantum registers 22 can be made to hold several data values for the quantum iterative optimization algorithm by subdividing the registers 22 into several registers or sub-registers.

The classical computer memory 14 holds the classical program and instructs the quantum computing system 10 components controlling each qubit what to do at each step. The qubits are interconnected. The classical program defines “quantum gates”, for example classical logic gates such as “AND” and “OR” gates, but quantum gates can be considered more akin to instructions in a classical computer. The instructions in the classical program instructing the quantum computing system 10 components controlling each qubit may be developed using the Controlled NOT (CNOT) gate, Hadamard and other suitable gates.

In the following description, the SIR model for epidemics will be used by way of describing the principles of the invention and as an example of one physical or real-world system to which the method and quantum computing system of the invention may be applied.

The SIR model describes the dynamics of infectious diseases. The SIR model divides the population into compartments, regions, or spaces where each compartment, region or space is expected to have the same characteristics.

In the following description, a reference to “region” is to be taken also as a reference to “compartment” and/or “space”.

In the SIR model, S is the number of individuals in a population susceptible to infection, I is the number of infected individuals in said population and R is the number of recovered individuals in said population. Consequently, the SIR model has three dependent variables.

To model the dynamics of an infectious disease outbreak in the population, 3 differential equations derived from respective non-linear equations defining the changes for each of S, I and R are required. In this case, the number c of dependent variables and thus the number c of non-linear equations is 3, i.e., c=3. The differential equations derived from the 3 respective non-linear equations comprise:

$$\frac{dS}{dt} = - \frac{β IS}{N}$$

$$\frac{dI}{dt} = \frac{β IS}{N} = - γ I$$

$$\frac{dR}{dt} = γ I$$

where N is the total of the population;
β is the average number of contacts per person per unit of time; and
γ is the probability of an infected case recovering and moving into the resistant phase.

It can be seen therefore that S, I and R comprise the dependent variables of the SIR model whilst time comprises an independent variable of the model. β and γ comprise some parameters of the SIR model on which the non-linear equations are based.

Calculating changes in the S, I and R dependent variables with respect to time using classical computers is complex and time consuming.

The invention therefore provides a Quantum Susceptible-Infectious-Recovered (QSIR) model for epidemics. The Quantum SIR is specially designed to regain the SIR model parameters. It can reduce the computational complexity and can be used to simulate the real situation and make future predictions for the values of one or more of S, I and R with respect to time.

Take by way of example the situation where the birth rate in a region is α, the mortality rate in the region is μ, a unit of time patient infection of susceptible probability is β, a per unit time will cure the disease probability is γ. The plurality of parameters comprises a parameters' vector (α, μ, β, γ) of the SIR model. In respect of β, assume that the number of susceptible persons that a patient can infect per unit time at t is proportional to the total number of susceptible persons in the environment (t), then we assume that this proportional coefficient is β. Also, suppose that at time t, the number of people removed from the infected person per unit time is proportional to the number of patients, then the proportional coefficient is γ.

For ease of description, the first-order nonlinear ordinary differential equations (ODEs) of the SIR model in the j-th region can be given by polynomials:

fⱼ₁(Sⱼ(t),Iⱼ(t),Rⱼ(t)),fⱼ₂(Sⱼ(t),Iⱼ(t),Rⱼ(t)),fⱼ₃(Sⱼ(t),Iⱼ(t),Rⱼ(t)).

Then, the SIR model for n regions in parallel can be calculated in the quantum system, i.e., the quantum computing system 10, assuming that the SIR model of different regions at the same time is given according to the below plurality of non-linear equations:

$${S}_{j}^{′} = {S}_{j} ( t + 1 ) - {S}_{j} ( t ) = \frac{{dS}_{j} ( t )}{dt} = α {N}_{j} ( t ) - β {S}_{j} ( t ) {I}_{j} ( t ) - μ {S}_{j} ( t )$$

$${I}_{j}^{′} = {I}_{j} ( t + 1 ) - {I}_{j} ( t ) = \frac{{dI}_{j} ( t )}{dt} = β {S}_{j} ( t ) {I}_{j} ( t ) - γ {I}_{j} ( t ) - μ {I}_{j} ( t )$$

$${R}_{j}^{′} = {R}_{j} ( t + 1 ) - {R}_{j} ( t ) = \frac{{dR}_{j} ( t )}{dt} = γ {I}_{j} ( t ) - μ {R}_{j} ( t )$$

where j∈{1, 2, . . . }, j signifying the region.

The method of the invention comprises transforming the plurality of non-linear equations for the SIR model into the following differential equations where the non-linear terms are defined by polynomials:

$$\frac{{dS}_{1} ( t )}{dt} = {f}_{11} ( {S}_{1} ( t ) , {I}_{1} ( t ) , {R}_{1} ( t ) )$$

$$⋮$$

$$\frac{{dS}_{n} ( t )}{dt} = {f}_{n 1} ( {S}_{n} ( t ) , {I}_{n} ( t ) , {R}_{n} ( t ) )$$

$$\frac{{dI}_{1} ( t )}{dt} = {f}_{12} ( {S}_{1} ( t ) , {I}_{1} ( t ) , {R}_{1} ( t ) )$$

$$⋮$$

$$\frac{{dI}_{n} ( t )}{dt} = {f}_{n 2} ( {S}_{n} ( t ) , {I}_{n} ( t ) , {R}_{n} ( t ) )$$

$$\frac{{dR}_{1} ( t )}{dt} = {f}_{13} ( {S}_{1} ( t ) , {I}_{1} ( t ) , {R}_{1} ( t ) )$$

$$⋮$$

$$\frac{{dR}_{n} ( t )}{dt} = {f}_{n 3} ( {S}_{n} ( t ) , {I}_{n} ( t ) , {R}_{n} ( t ) )$$

Consequently, the transforming step comprises transforming the plurality of non-linear equations into differential equations for n regions in parallel in the quantum computing system 10. It will be seen that the number c of the plurality of non-linear equations is equal to the number of the plurality of dependent variables and that the number of the differential equations comprises a product (c×n) of the number c of the plurality of non-linear equations and the number n of regions in parallel. The quantum computing system 10 comprises a (cn+m) level quantum system where c is equal to the number of dependent variables or non-linear equations, n is equal to the number of regions in parallel of the quantum system, and m is equal to the number of ancilla qubits in the quantum system. More specifically, for the SIR model, the quantum computing system 10 comprises a (3n+m) level quantum system.

In the QSIR model as described, a region could comprise a country, a province, a city, a town, or the like. The QSIR model assumes there are n regions such that the differential equation of the nth region vulnerable or susceptible (S) to infection is expressed as differential equation “fn1”, the differential equation of the infected (I) is expressed as “fn2”, and the differential equation of the cured or recovered (R) is expressed as “fn3”. Consequently, fl1 is the differential equation expression of the susceptible (S) to infection in a first region of the n regions. fl2 is the differential equation expression of the infected (I) in said first region and fl3 is the differential equation expression of the recovered (R) in said first region.

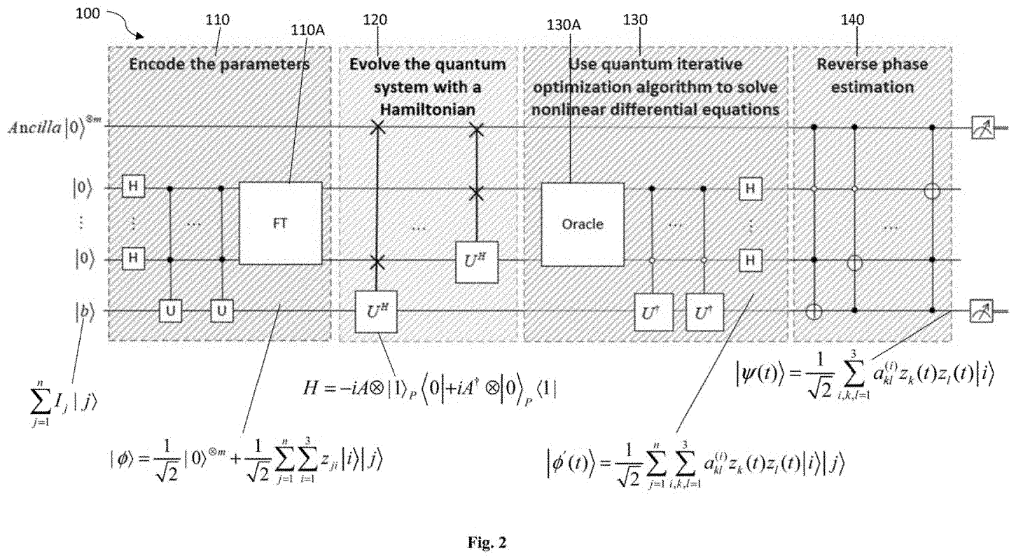

Significant further steps of the method 100 of the invention are illustrated in the quantum gate diagram of FIG. 2. FIG. 2 illustrates steps of the method 100 performed in the quantum computer 18 under control of the classical program stored in the memory 14 of the classical computer 12.

This structure combines the quantum Fourier algorithm, the quantum Hamiltonian algorithm, and the quantum iterative optimization algorithm to construct an SIR model that can predict future disease trends.

The inputs to the computing system 10 comprise the number S of susceptible persons. the number I of infected person and the number R of recovered persons at the time or moment of initialization leading to the first step 110 of the method 100 of encoding the parameters. The known parameters in the SIR model are encoded in step 110 into amplitudes in the quantum state.

The step 110 comprises encoding the differential equation polynomials as probability amplitudes of a quantum state of the quantum computing system 10. This may comprise encoding S(t₀), I(t₀) and R(t₀) as the probability amplitudes of the quantum state of the (3n+m) level quantum system for the SIR model. Using:

fⱼᵢ(zⱼ₁(t),zⱼ₂(t),zⱼ₃(t))(zⱼ₁(t)=S(t),zⱼ₂(t)=I(t),zⱼ₃(t)=R(t)

to represent the i-th equation of the SIR model for the j-th region, Zji is used to store the value of Zji(t) for the j-th region. To ensure that the quantum state is normalized, the following equality must be observed:

Σⱼ₌₁ⁿΣᵢ₌₁³|zⱼᵢ|²=1.

The quantum state is normalized because the modulus of the quantum amplitude represents the probability of a particle appearing at a certain point in Hilbert space.

The quantum state can then be expressed as:

$$\vert ϕ 〉 = \frac{1}{\sqrt{2}} {\vert 0 〉}^{⊗ m} + \frac{1}{\sqrt{2}} {\sum }_{j = 1}^{n} {\sum }_{i = 1}^{3} {z}_{ji} \vert i 〉 \vert j 〉 . \\ where {\sum }_{j = 1}^{n} {\vert {z}_{j} \vert}^{2} = 1 ;$$

and

where zⱼᵢ|i [symbol] |j [symbol] is the probability amplitude of the quantum state of the quantum system for the j-th region or space of the quantum system and for the i-th dependent variable.

FIG. 2 illustrates the processing of the I independent variable of the QSIR model where:

$${\sum }_{j = 1}^{n} {I}_{j} \vert j 〉 .$$

Step 110 includes a quantum Fourier Transform algorithm sub-step 110A which transforms the input quantum state into a superposition state of the set quantum ground state. This sub-step 110A is denoted by box “FT” in the quantum gate diagram of FIG. 2.

In a next step 120 of the method 100, the quantum system is evolved into a new quantum system comprising message qubits and at least one ancilla qubit. This is preferably done using a Hamiltonian in which the original parameters are evolved to the Hamiltonian for use in subsequent calculations. The Hamiltonian is based on the plurality of parameters of the QSIR model, i.e., on the parameter vector of the QSIR model. The Hamiltonian is used to represent the change of the quantum system, describe its energy value and characteristics, and is novel in the present invention in the selection of the initial state preparation, which can efficiently simulate the equations of the SIR model and which can be evolved in accordance with the invention into the corresponding quantum system for finding solutions to the differential equations.

The evolving step 120 preferably includes initializing the quantum system in the state |ϕ [symbol] ϕ [symbol] |0 [symbol] and then evolving the quantum system into the new quantum system according to:

$$\begin{matrix}\vert Ψ 〉 = {e}^{iòH} \vert ϕ 〉 \vert ϕ 〉 \vert 0 〉 \\ = {\sum }_{j = 0}^{∞} \frac{{( iòH )}^{j}}{j !} \vert ϕ 〉 \vert ϕ 〉 \vert 0 〉\end{matrix}$$

$$where$$

$$H = - iA ⊗ {\vert 1 〉}_{P} 〈 0 \vert + {iA}^{†} ⊗ {\vert 0 〉}_{P} 〈 1 \vert ;$$

and A is utilized to set up the Hamiltonian.

The method 100 includes the novel step 130 of utilizing a quantum iterative optimization algorithm to solve the plurality of differential equations according to:

$$\vert {ϕ}^{′} ( t ) 〉 = \frac{1}{\sqrt{2}} {\sum }_{j = 1}^{n} {\sum }_{i = 1}^{3} {f}_{ji} ( {z}_{ji} ( t ) ) \vert i 〉 \vert j 〉 = \frac{1}{\sqrt{2}} {\sum }_{j = 1}^{n} {\sum }_{i , k , l = 1}^{3} {a}_{kl}^{( i )} {z}_{k} ( t ) {z}_{l} ( t ) \vert i 〉 \vert j 〉 .$$

Step 130 includes, prior to solving the plurality of differential equations, the steps of selecting a small step size h, iterating the map zⱼ [symbol] zⱼ+hzⱼ′=zⱼ+hfⱼ(z); and integrating the quantum system using Euler's method to prepare:

|ϕ(t+h) [symbol] =|ϕ(t) [symbol] +h|ϕ′(t) [symbol] +O(h²).

In step 130, the Oracle box denoted by numeral 130A in FIG. 2 bases step 130 on a specific execution time data to determine a decision function.

The method 100 may include the step 140 of performing a reverse phase estimation operation on the message qubits prior to a step of measuring the at least one ancilla qubit to determine or calculate a value for one or more of the dependent variables S, I, R with respect to the at least one independent variable time. The step 140 solves the decision function to output predicted numbers susceptible (S), infected (I), and recovered (R) at time t based on the SIR model.

The step 140 of performing a reverse phase estimation operation on the message qubits comprises performing the reverse phase estimation on the j-th pair of message qubits, |ϕⱼ [symbol] |ϕⱼ [symbol] |0 [symbol] , according to:

|ϕ [symbol] ϕ [symbol] |0 [symbol] [symbol] \sqrt{ [symbol]−ϵ²H²}|ϕ [symbol] ϕ [symbol] |0 [symbol] +iϵH|ϕ [symbol] ϕ [symbol] |0 [symbol] .

The values of one or more of the dependent variables S, I, R with respect to the independent variable time for a region n of the quantum system at a point in time t is determined or calculated according to:

$$\vert ψ ( t ) 〉 = \frac{1}{\sqrt{2}} {\sum }_{i , k , l = 1}^{3} {a}_{kl}^{( i )} {z}_{k} ( t ) {z}_{l} ( t ) \vert i 〉 .$$

In the method 100, at least the encoding step 110, the evolving step 120, the quantum iterative optimization algorithm step 130 is implemented in the one or more quantum processors.

The apparatus described above may be implemented at least in part in software. Those skilled in the art will appreciate that the apparatus described above may be implemented at least in part using general purpose computer equipment or using bespoke equipment.

Here, aspects of the methods and apparatuses described herein can be executed on any apparatus comprising the communication system. Program aspects of the technology can be thought of as “products” or “articles of manufacture” typically in the form of executable code and/or associated data that is carried on or embodied in a type of machine-readable medium. “Storage” type media include any or all of the memory of the mobile stations, computers, processors or the like, or associated modules thereof, such as various semiconductor memories, tape drives, disk drives, and the like, which may provide storage at any time for the software programming. All or portions of the software may at times be communicated through the Internet or various other telecommunications networks. Such communications, for example, may enable loading of the software from one computer or processor into another computer or processor. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible non-transitory “storage” media, terms such as computer or machine “readable medium” refer to any medium that participates in providing instructions to a processor for execution.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A method of determining or calculating a value of a dependent variable with respect to a value of an independent variable in a physical system defined or modelled by at least one independent variable, a plurality of dependent variables and a plurality of parameters associated with said plurality of dependent variables, said plurality of dependent variables being defined by a plurality of non-linear equations, each non-linear equation being based on at least one of the plurality of parameters, the method comprising the steps of:
transforming the plurality of non-linear equations into differential equations whose non-linear terms are defined by polynomials;
encoding the polynomials as probability amplitudes of a quantum state of a quantum system;
evolving the quantum system into a new quantum system comprising message qubits and at least one ancilla qubit;
utilizing a quantum iterative optimization algorithm to solve the plurality of differential equations; and
measuring the at least one ancilla qubit to determine or calculate a value for at least one of the dependent variables with respect to the at least one independent variable.

2. The method of claim 1, wherein, prior to the measuring step, the method includes the step of performing a reverse phase estimation operation on the message qubits.

3. The method of claim 1, wherein the transforming step includes transforming the plurality of non-linear equations into differential equations for n regions or spaces in parallel of the quantum system.

4. The method of claim 1, wherein the transforming step includes transforming the plurality of non-linear equations into ordinary differential equations for n regions or spaces in parallel of the quantum system.

5. The method of claim 3, wherein a number c of the plurality of non-linear equations is equal to a number of the plurality of dependent variables and a number of the differential equations comprises a product (c×n) of the number c of the plurality of non-linear equations and the number n of regions or spaces in parallel of the quantum system.

6. The method of claim 1, wherein the quantum system is a (cn+m) level quantum system, where c is equal to the number of dependent variables or non-linear equations, n is equal to the number of regions or spaces in parallel of the quantum system, and m is equal to the number of ancilla qubits.

7. The method of claim 1, wherein the encoding step expresses the quantum state as:
$$\vert ϕ 〉 = \frac{1}{\sqrt{2}} {\vert 0 〉}^{⊗ m} + \frac{1}{\sqrt{2}} {\sum }_{j = 1}^{n} {\sum }_{i = 1}^{c} {z}_{ji} \vert i 〉 \vert j 〉 , \\ where {\sum }_{j = 1}^{n} {\vert {z}_{j} \vert}^{2} = 1 ;$$
and
where zⱼᵢ|i [symbol] |j [symbol] is the probability amplitude of the quantum state of the quantum system for the j-th region or space of the quantum system and for the i-th dependent variable; and
where c is equal to the number of dependent variables or non-linear equations and n is equal to the number of regions or spaces in parallel of the quantum system.

8. The method of claim 7, wherein, to ensure the quantum state is normalized, the following equality is applied to the probability amplitudes of the quantum state of the quantum system:
Σⱼ₌₁ⁿΣᵢ₌₁^{c}|zⱼᵢ|²=1.

9. The method of claim 1, wherein the evolving step comprises evolving the quantum system into a new quantum system comprising message qubits and at least one ancilla qubit with a Hamiltonian.

10. The method of claim 9, wherein the Hamiltonian is based on the plurality of parameters.

11. The method of claim 9, wherein the evolving step comprises:
initializing the quantum system in the state |ϕ [symbol] |ϕ [symbol] |0 [symbol] ; and
evolving the quantum system into the new quantum system according to:
$$\begin{matrix}\vert Ψ 〉 = {e}^{iòH} \vert ϕ 〉 \vert ϕ 〉 \vert 0 〉 \\ = {\sum }_{j = 0}^{∞} \frac{{( iòH )}^{j}}{j !} \vert ϕ 〉 \vert ϕ 〉 \vert 0 〉\end{matrix}$$
$$where$$
$$H = - iA ⊗ {\vert 1 〉}_{P} 〈 0 \vert + {iA}^{†} ⊗ {\vert 0 〉}_{P} 〈 1 \vert ;$$
and A is utilized to set up the Hamiltonian.

12. The method of claim 11, wherein the step of utilizing a quantum iterative optimization algorithm comprises solving the plurality of differential equations according to:
$$\vert {ϕ}^{′} ( t ) 〉 = \frac{1}{\sqrt{2}} {\sum }_{j = 1}^{n} {\sum }_{i = 1}^{c} {f}_{ji} ( {z}_{ji} ( t ) ) \vert i 〉 \vert j 〉 = \frac{1}{\sqrt{2}} {\sum }_{j = 1}^{n} {\sum }_{i , k , l = 1}^{c} {a}_{kl}^{( i )} {z}_{k} ( t ) {z}_{l} ( t ) \vert i 〉 \vert j 〉 .$$

13. The method of claim 12, wherein the step of utilizing a quantum iterative optimization algorithm includes, prior to solving the plurality of differential equations, the steps of:
selecting a small step size h;
iterating the map zⱼ [symbol] zⱼ+hzⱼ′=zⱼ+hfⱼ(z); and
integrating the quantum system using Euler's method.

14. The method of claim 2, wherein the step of performing a reverse phase estimation operation on the message qubits comprises performing the reverse phase estimation on the j-th pair of message qubits, |ϕⱼ [symbol] |ϕⱼ [symbol] |0 [symbol] , according to:

|ϕ [symbol] ϕ [symbol] |0 [symbol] [symbol] \sqrt{ [symbol]−ϵ²H²}|ϕ [symbol] ϕ [symbol] |0 [symbol] +iϵH|ϕ [symbol] ϕ [symbol] |0 [symbol] .

15. The method of claim 14, wherein values of one or more of the dependent variables with respect to the independent variable comprising time for a region or space n of the quantum system at a point in time t are determined or calculated according to:
$$\vert ψ ( t ) 〉 = \frac{1}{\sqrt{2}} {\sum }_{i , k , l = 1}^{c} {a}_{kl}^{( i )} {z}_{k} ( t ) {z}_{l} ( t ) \vert i 〉 .$$

16. The method of claim 1, wherein the physical system comprises a Susceptible-Infectious-Recovered (SIR) Model for epidemics where the at least one independent variable is time and the dependent variables comprise a number (S) of individuals in a population susceptible to infection, a number (I) of infected individuals in said population and a number (R) of recovered individuals in said population.

17. The method of claim 16, wherein the plurality of parameters comprises any one or any combination of a birth rate (α) of the population, a mortality rate (μ) of the population, a unit of time patient infection of susceptible probability (β), a per unit time will cure the disease probability (γ), the plurality of parameters comprising a parameters' vector (α, μ, β, γ) of the SIR model.

18. The method of claim 1, wherein the quantum iterative optimization algorithm is implemented in a quantum processor.

19. A computing system comprising:
a quantum computer comprising:
a quantum computer controller; and
one or more quantum processors;
a classical computer comprising:
a memory storing machine-readable instructions; and
a processor for executing the machine-readable instructions such that, when the processor executes the machine-readable instructions, it configures the classical computer to model a physical system defined by at least one independent variable, a plurality of dependent variables and a plurality of parameters associated with said plurality of dependent variables, said plurality of dependent variables being defined by a plurality of non-linear equations, each non-linear equation being based on at least one of the plurality of parameters to determine or calculate a value of one or more of said dependent variables with respect to a value of one of more of said independent variables in said physical system; and
to implement the steps in said computing system of:
transforming the plurality of non-linear equations into differential equations whose non-linear terms are defined by polynomials;
encoding the polynomials as probability amplitudes of a quantum state of a quantum system;
evolving the quantum system into a new quantum system comprising message qubits and at least one ancilla qubit;
utilizing a quantum iterative optimization algorithm to solve the plurality of differential equations; and
measuring the at least one ancilla qubit to determine or calculate a value for one of the dependent variables with respect to the at least one independent variable;
wherein the at least the utilizing step is performed by the one or more quantum processors.

20. A non-transitory computer-readable medium storing machine-readable instructions for a computing system comprising a quantum computer, which comprises a quantum computer controller and one or more quantum processors, and a classical computer comprising said non-transitory computer-readable medium and a processor, wherein, when the machine-readable instructions are executed by said processor it configures the classical computer to model a physical system defined by at least one independent variable, a plurality of dependent variables and a plurality of parameters associated with said plurality of dependent variables, said plurality of dependent variables being defined by a plurality of non-linear equations, each non-linear equation being based on at least one of the plurality of parameters to determine or calculate a value of one or more of said dependent variables with respect to a value of one of more of said independent variables in said physical system; and
to implement the steps in said computing system of:
transforming the plurality of non-linear equations into differential equations whose non-linear terms are defined by polynomials;
encoding the polynomials as probability amplitudes of a quantum state of a quantum system;
evolving the quantum system into a new quantum system comprising message qubits and at least one ancilla qubit;
utilizing a quantum iterative optimization algorithm to solve the plurality of differential equations; and
measuring the at least one ancilla qubit to determine or calculate a value for one of the dependent variables with respect to the at least one independent variable;
wherein the at least the utilizing step is performed by the one or more quantum processors.
